(19) United States
(12) Patent Application Publication
(10) Pub. No.: US 2003/0165456 A1
(43) Pub. Date: Sep. 4, 2003

(54) Skin care compositions and methods of improving aesthetic appearance of skin using same

(75) Inventors: John Duffy, West Milford, NJ (US); Brent Slobody, New City, NY (US); Susan Wade, Yonkers, NY (US)

Correspondence Address: Charles N.J. Ruggiero, Esq., Ohlandt, Greeley, Ruggiero & Perle, L.L.P., One Landmark Square, 10th Floor, Stamford, CT, 06901-2682, US

(73) Assignee: AVON PRODUCTS, NEW YORK, NY

(21) Appl. No.: 10/145,591

(22) Filed: May 14, 2002

Related U.S. Application Data
(60) Provisional application No. 60/290,875, filed on May 14, 2001.

(51) Int. Cl. A61K 35/78; A61K 31/728; A61K 7/00

(52) U.S. Cl. 424/74; 424/757; 424/764; 424/776; 514/54

(57) ABSTRACT

The present invention relates to cosmetic compositions that may be topically applied to skin, hair and nails for improving the aesthetic appearance thereof. The present invention also relates to compositions that may be topically applied to skin, hair and nails for ameliorating, forestalling, reducing, preventing, and/or treating the signs of aging due to extrinsic and/or intrinsic factors.

### RELATED APPLICATION

This application claims priority in U.S. application Ser. No. 60/290,875, filed on May 14, 2001.

### BACKGROUND OF THE INVENTION

### FIELD OF THE INVENTION

The present invention relates to cosmetic compositions that may be topically applied to skin, hair and/or nails for improving the aesthetic appearance thereof. The present invention also relates to compositions that may be topically applied to skin, hair and/or nails for ameliorating, forestalling, reducing, preventing, and/or treating the signs of aging due to extrinsic and/or intrinsic factors.

### SUMMARY OF THE INVENTION

It is another object of the present invention to provide cosmetic compositions for topical application to skin, hair or nails that improve the aesthetic appearance thereof.

It is an object of the present invention to provide such cosmetic compositions that ameliorate, forestall, reduce, prevent, and/or treat the diminished aesthetic appearance of skin, hair and/or nails due to one or more stress factors.

These and other objects and advantages of the present invention are achieved by one or more cosmetic compositions of the present invention that include: a cosmetically acceptable vehicle, and a combination of at least two of the following ingredients: sunflower seed extract; pomegranate extract; soy bean extract; grape extract; panthenol; hyaluronic acid; phytol, isophytol or a mixture thereof; and cucumber extract. The combination is present in an amount effective to improve the aesthetic appearance of skin, hair and/or nails.

### DETAILED DESCRIPTION OF THE INVENTION

The present invention relates to a combination of ingredients that that may be either applied directly to skin, hair and/or nails or incorporated into cosmetic compositions for topical application thereto.

The present invention provides a “benefit reservoir” that provides skin, hair and/or nails with the weapons it needs to respond to challenges from intrinsic and/or extrinsic sources, such as environmental stressors, biological stressors and/or lifestyle stressors. These stressors decrease the overall health and, thus, aesthetic appearance of the skin, hair and/or nails. In particular, the present invention can be used to ameliorate, forestall, reduce, prevent, and/or treat the diminished aesthetic appearance of skin, hair and/or nails due to one or more of the following stress factors.

1. Environmental Stress Factors

Examples of environmental stress factors (or aggressors) include, but are not limited to, ultraviolet radiation, pollutants, smog, ozone, free radicals, tobacco smoke, wind and dry environmental conditions.

2. Biological Stress Factors

Endogenous changes and/or fluctuations in hormonal levels (e.g., peri-menopause, menopause, menstruation) or negative health factors (e.g., inappropriate glucose utilization/metabolism, such as diabetes).

3. Lifestyle Stress Factors

Examples of lifestyle stress factors include, but are not limited to: smoking; sunbathing; and inadequate diet from a balanced, nutritional perspective. Lifestyle stress factors can also include ever busier schedules that no longer allow consumers to take the preventative measures, such as applying moisturizers and/or removing make-up, necessary to decrease the negative impact various stress factors have on the appearance of skin, hair and/or nails.

The complexity of modern life is such that most consumers experience the adverse effect of multiple stress factors from more than one, or even all three, of the above-described stressors. These multiple stress factors constantly assault and, thus, compromise the integrity, health and overall aesthetic appearance of the skin, hair and/or nails. One of the many benefits of the present invention is due to the self-regulating, multi-pronged approach to ameliorate, forestall, reduce, prevent, and/or treat these stress factors.

As mentioned above, the present invention provides a “benefits reservoir” from which the skin, hair and/or nails can extract the specific benefit it needs at the specific time of need to cope with the ever-changing demands of life and its stress factors.

Specifically, to compensate for the above-described stress factors, the compositions of the present invention include one or more of the following benefit components:

1. Collagen Enhancing Component

Loss of firmness and elasticity of skin, in particular, is a result of photoaging and chronological aging, both of which trigger degradation of collagen. Specifically, degradation of collagen manifests as abnormal cross-linking of collagen fibers that cause the collagen fibers to be rigid and less flexible. Accordingly, it is preferred that the compositions of the present invention include a collagen-enhancing component, which may include a collagenase or elastase inhibitor. Examples of suitable collagen enhancing components include, but are not limited to, sunflower seed extract, fatty acids, such as oleic acid, perinaric acid, and honeysuckle extract (Lonicera caprifolium). Preferably, the collagen-enhancing component includes sunflower seed extract. More preferably, the collagen-enhancing component is sunflower seed extract.

2. Bio-balancing/Hormonal Balancing Component

Examples of suitable bio-balancing/hormonal balancing components include, but are not limited to, pomegranate extract and/or soybean extract. More preferably, the bio-balancing/hormonal balance component includes both pomegranate extract and soybean extract. While not wishing to be bound by any one theory, the inventors believe that the phytoestrogens in natural extracts, which are preferably pomegranate extract and/or soy bean extract, provide the benefit reservoir with the ability to compensate for hormonal changes that occur in women at all ages, either due to daily or monthly hormonal cycles. More importantly, women in their peri- or post-menopausal years have diminished internal reservoirs of hormones. Accordingly, they can especially benefit from the present invention, particularly since diminished and/or fluctuating hormone levels can lead to skin changes, such as dry, flaky skin, discoloration/blotchiness, wrinkles, and loss of firmness and elasticity.

3. Antioxidant/Free Radical Scavenging Component

Compositions of the present invention preferably include an antioxidant/free radical scavenging component. Pollution, sun, tobacco smoke, among other detrimental influences, can lead to the generation of free radicals. Free radicals are highly damaging molecules that negatively affect the skin, hair and/or nails at many levels. Although antioxidants/free radical scavengers known in the art are useful in the present invention, it is preferred that the antioxidant free radical scavenging component of the present invention includes or is a grape extract.

4. Moisture Replenishing Component

Preferably, the present invention includes a moisture-replenishing component. Dry, flaky skin can result from the onslaught of more than one stress factor. For example, extrinsic factors, such as low humidity, sun exposure and/or cleansers, result in moisture loss from skin, hair and/or nails. Hormonal fluctuations/changes, particularly during menstruation, and perimenopause/postmenopause years, are also associated with increased epidermal water loss from the skin. Examples of suitable moisture replenishing components include, but are not limited to, hyaluronic acid, vitamin B5 (panthenol) and vitamin B6 (pyridoxine, pyridoxal, pyridoxamine). Preferably, the moisture-replenishing component includes hyaluronic acid. More preferably, the moisture-replenishing component is a mixture of hyaluronic acid and panthenol.

5. Antiaging Regulation/Age Defense Component

The antiaging regulation/age defense component preferably includes phytol and/or sunflower seed extract. More preferably, this component is a combination of phytol and sunflower seed extract. Phytol is enzymatically converted in the skin to phytanic acid. It has been discovered that phytol induces and promotes the biosynthesis and/or bioactivity of endogenous chemicals when topically applied to the skin. The discovery of surprising, unexpected benefits provided by phytol/phytanic acid on skin is described in commonly owned U.S. Provisional Patent Applications Serial No. 60/190,988, filed on Mar. 21, 2000 and commonly owned U.S. patent application Ser. No. 09/724,356 filed Nov. 28, 2000. Both foregoing patent applications are incorporated herein by reference.

Significantly, it has now been discovered that phytanic acid activates or “upregulates” PPAR responsive genes. Upregulation of PPAR responsive genes has been documented to improve the skin barrier function and, hence, moisture retention by skin. As a result, both extrinsic and intrinsic skin aging are reduced by topically applying compositions containing phytanic acid precursors, such as phytol. In addition, adverse or deleterious effects of retinoids, such as dry skin, flaky skin and/or reddened (i.e. sensitized) skin, are minimized by retention of moisture in the skin due to PPAR receptor upregulation.

Moreover, phytanic acid is a recognized RXR antagonist competing with retinoids, such as retinol, at the receptor site. Thus, phytanic acid will bind and activate available RXR sites, leaving only RAR sites available for binding and activating with a lesser or smaller level of retinoids. Thus, combining phytanic acid with one or more retinoids enhances the efficacy of the retinoids by increasing the number of RAR sites activated absent the presence of phytanic acid.

The discovery of surprising, unexpected skin care benefits provided by sunflower seed extract has been described in commonly owned U.S. patent application Ser. Nos. 60/190,988 and 09/461,449 and PCT Application No. US/00/33776. These two applications are incorporated herein by reference.

6. Anti-Inflammatory/Soothing Component

Preferably, the anti-inflammatory/soothing component includes or is cucumber extract. Intrinsic and extrinsic stress factors can cause skin to feel irritated or sensitive. An anti-inflammatory/soothing component included in the benefits reservoir serves to prevent, reduce or ameliorate such irritation or sensitivity.

Compositions of the present invention preferably include at least one benefit component from at least two groups of benefit components. Most preferably, compositions of the present invention include at least one benefit component from each of group benefit components. One example of a most preferred benefit reservoir combination according to the present invention includes: elastinase/collagenase inhibiting agent sunflower seed extract; pomegranate extract and soy bean extract as the bio-balancing/hormonal balancing component; grape extract as the antioxidant/free radical scavenging component; panthenol and hyaluronic acid as the moisture replenishing component; phytol as the antiaging regulation/time defense component; and cucumber extract as the anti-inflammatory/soothing component. Table 1 below sets forth the preferred and most preferred parameters for this most preferred benefits reservoir combination based on percentage by weight (wt %) of the total weight of the composition.

TABLE 1
Preferred Composition

| | | | More | Most | |
|---|---|---|---|---|---|
| | | Preferred | Preferred | Preferred | |
| | Range | Range | Range | Range | Optimally |
| Ingredient | (wt %) | (wt %) | (wt %) | (wt %) | (wt %) |
| Sunflower | about | about 0.0001 | about 0.0001 | about 0.001 | about 0.001 |
| Seed Extract | 0.0001 to | to about 0.5 | to about 0.05 | to about 0.5 | to about 1 |
| | about 1 | | | | |
| Pome- | about | about 0.0005 | about 0.0075 | about 0.001 | about 0.001 |
| granate | 0.0001 to | to about 1 | to about 0.15 | to about 0.15 | to about 0.1 |
| Extract | about 5 | | | | |
| Soy Bean | about | about 0.005 to | about 0.001 to | about 0.001 | about 0.001 |
| Extract | 0.0001 to | about 0.5 | about 0.1 | to about 0.05 | to about 0.01 |
| | about 1 | | | | |
| Grape | about | about 0.0075 | about 0.001 to | about 0.001 | about 0.001 |
| Extract | 0.0001 to | to about 0.25 | about 0.1 | to about 0.15 | to about 0.1 |
| | about 1 | | | | |
| Panthenol | about | about 0.0001 | about 0.001 to | about 0.001 | about 0.001 |
| | 0.0001 to | to about 0.5 | about 0.1 | to | to about 0.1 |
| | about 5 | | | about 0.05 | |
| Hyaluronic | about | about 0.0005 | about 0.0007 | about 0.001 | about 0.001 |
| Acid | 0.0001 to | to about 0.05 | to about 0.01 | to about | |
| | about 0.1 | | | 0.01 | |
| Phytol and/or | about | about 0.001 to | about 0.001 to | about 0.001 | about 0.001 |
| Isophytol | 0.0001 to | about 1.5 | about 1.0 | to about 0.5 | to about 1 |
| | about 5 | | | | |
| Cucumber | about | about 0.0005 | about 0.007 to | about 0.001 | about 0.01 |
| Extract | 0.005 to | to about 4 | about 4 | to about 0.1 | |
| | about 5.0 | | | | |
| Cosmetic- | qs | qs | qs | qs | Qs |
| ally | | | | | |
| Acceptable | | | | | |
| Vehicle | | | | | |

Preferably, the sunflower seed extract is a water-soluble extract of Helianthus annus seed. The sunflower seed extract may be provided in a water or hydrophilic solvent. An example of a preferred sunflower seed extract is available from SILAB under the trade name ANTIGLYSKIN, which contains sunflower seed extract in a water carrier.

Preferably, the pomegranate extract is extracted from the pomegranate fruit. More preferably, the pomegranate extract is a hydroglycolic extract obtained from the whole fruit by a water-based solvent. Most preferably, the water-based solvent is 50:50 propylene glycol:water. An example of such a preferred pomegranate extract is available from Active Organics, Inc. under the trade name ACTIPHYTE OF POMEGRANATE.

Preferably, the soybean extract has at least about 8 wt % isoflavanoids, based on the total wt % of the extract. More preferably, the soybean extract has at least about 12 wt % isoflavanoids, based on the total wt % of the extract. The soybean extract is preferably obtained from the beans of Glycine soya. An example of a preferred soybean extract is available from Shouten USA under the trade name SOYLIFE-150.

Preferably, the grape extract is extracted from the genus Vitis vinifera. It is preferred that the grape extract is obtained from the leaves, flowers and/or seed of the grape plant. It is most preferred that the grape extract is obtained from the leaves of the grape plant. An example of a preferred extract is available from Indena SA under the trade name PHYTELENE EG-218.

The panthenol may be in the form of DL-panthenol. An example of a preferred panthenol is available from C.P. PhibroChem, Div. of Philipp Brothers Chem. under the tradename DL-PANTHENOL RACEMIC PANTHENOL FCC GRADE.

Hyaluronic acid can be incorporated either as hyaluronic acid or as its salt. One preferred form of hyaluronic acid is sodium salt of hyaluronic acid. An example of such a preferred form of sodium salt of hyaluronic acid is available from Nikko Chem. under the tradename NIKKOL SODIUM HYALURONATE.

The phytol may be a blend of the cis and trans isomers of 3,7,11,15-tetramethylhexadec-2-en-l-ol, i.e., a blend of phytol and isophytol. An example of such a preferred blend of phytol/isophytol is available from BASF Corp. under the tradename PHYTOL.

The cucumber extract is preferably a hydroglycolic extract obtained from cucumber, which is preferably Cucumber sativis. The extract solvent is preferably a water-based solvent. More preferably, the extract solvent is a water/propylene glycol solvent. Most preferably, the extract solvent is at 75:25 water:propylene glycol ratio. An example of such a most preferred extract is available from Vege-Tech International under the tradename CUCUMBER VT-01 in 25% proplylene glycol.

In particular, compositions of the present invention can be topically applied to improve the aesthetic appearance of skin, hair and/or nails. The improvement to the aesthetic appearance of skin will be demonstrated as one of the following:

1. reducing dermatological aging;

2. decreasing skin fragility;

3. ameliorating and/or reversing loss of collagen and/or elastin;

4. ameliorating skin atrophy;

5. improving skin firmness/plumpness;

6. improving skin texture;

7. reducing the appearance/depth of lines and wrinkles, including fine lines and wrinkles;

8. improving skin tone/tautness;

9. decreasing pore size;

10. restoring skin luster/brightening/reduce sallowness;

11. minimizing signs of fatigue;

12. improving skin barrier function;

13. minimizing skin dryness;

14. reducing or ameliorating hyperpigmentation;

15. promoting/accelerating cell turnover;

16. enhancing skin thickness;

17. improving the appearance of skin contour;

18. increasing skin resiliency; and/or

19. minimizing skin discoloration.

The compositions of the present invention may also include one or more oxa acids, oxa diacids, sunscreens, vitamin E and/or vitamin E acetate, vitamin C and/or vitamin C palmitate, kelp sea extract, cyclic AMP, ivy glycolic extract, willow bark extract, oat beta glucan, hops extract, fennel extract, milk extract, jojoba oil, ginseng extract, among others, or any combinations thereof.

Preferably, the sunscreen is one or more of the following: oxybenzone, sulsiobenzone, dioxybenzone, menthyl anthranilate, para aminobenzoic acid (PABA), DEA methoxycinnamate, octocrylene, octyl methoxycinnamate, octyl salicylate, homomenthyl salicylate, octyl dimethyl PABA, TEA salicylate, titanium dioxide, zinc oxide, butylmethoxy dibenzoylmethane, 4-methyl benzilidene camphor, octyl triazone, terephthalydidene dicamphor sulfonic acid and salts thereof, ethyl PABA, 2-(2′hydroxy-5′-methylphenyl) benzotriazole, methylene bis-benzotriazolyl tetramethylbutylphenol, phenylbenzimidazole sulfonic acid, bis-octoxyphenol methoxphenyl triazine, and any combinations thereof.

Thus, the present invention improves the overall aesthetic appearance of skin, hair and/or nails by making skin, hair and/or nails appear more healthy, youthful, vital and/or vibrant.

The present invention having been described with particular reference to the preferred forms thereof, it will be obvious that various changes and modification may be made herein without departing from the spirit and scope of the present invention.

What is claimed is:

1. A composition comprising:
a cosmetically acceptable vehicle, and
a combination of at least two ingredients selected from the group consisting of sunflower seed extract; pomegranate extract; soy bean extract; grape extract; panthenol; hyaluronic acid; phytol, isophytol or a mixture thereof; and cucumber extract,
wherein the combination is present in an amount effective to improve the aesthetic and/or cosmetic appearance of skin, hair and/or nails.

2. The composition of claim 1, wherein the combination comprises at least three of the ingredients.

3. The composition of claim 1, wherein the combination comprises at least four of the ingredients.

4. The composition of claim 1, wherein the combination comprises at least five of the ingredients.

5. The composition of claim 1, wherein the combination comprises at least six of the ingredients.

6. The composition of claim 1, wherein the composition comprises at least seven of the ingredients.

7. The composition of claim 1, wherein the combination comprises about 0.001 wt % to about 0.1 wt % of the grape extract and about 0.01 wt % to about 0.1 wt % of the cucumber extract.

8. The composition of claim 7, wherein the combination further comprises about 0.001 to about 1 wt % of phytol, isophytol or the mixture.

9. The composition of claim 1, wherein the combination comprises about 0.001 wt % to about 0.01 wt % of the soy bean extract and about 0.01 wt % to about 0.1 wt % of the cucumber extract.

10. The composition of claim 9, wherein the combination further comprises about 0.001 to about 1 wt % of phytol, isophytol or the mixture.

11. A composition comprising:
sunflower seed extract;
pomegranate extract;
soy bean extract;
grape extract;
panthenol;
hyaluronic acid;
phytol, isophytol or a combination thereof;
cucumber extract, and
a cosmetically acceptable vehicle.

12. The composition of claim 11, wherein, based upon the total weight of the composition, the sunflower seed extract is present from about 0.000001 wt % to about 1 wt %; the pomegranate extract is present from about 0.0001 wt % to about 5 wt %; the soy bean extract is present from about 0.0001 wt % to about 5 wt %; the grape extract is present from about 0.0001 wt % to about 1 wt %; the panthenol is present from about 0.0001 wt % to about 5 wt %; the hyaluronic acid is present from about 0.0001 wt % to about 0.1 wt %; the phytol, isophytol or the combination thereof is present from about 0.0001 to about 5 wt %; and the cucumber extract is present from about 0.005 wt % to about 5 wt %.

13. The composition of claim 11, wherein, based upon the total weight of the composition, the sunflower seed extract is present from about 0.0001 wt % to about 0.5 wt %; the pomegranate extract is present from about 0.0005 wt % to about 1 wt %; the soy bean extract is present from about 0.005 wt % to about 0.5 wt %; the grape extract is present from about 0.0075 wt % to about 0.25 wt %; the panthenol is present from about 0.0001 wt % to about 0.5 wt %; the hyaluronic acid is present from about 0.0005 wt % to about 0.05 wt %; the phytol, isophytol or the combination thereof is present from about 0.001 to about 1.5 wt %; and the cucumber extract is present from about 0.0005 wt % to about 4 wt %.

14. The composition of claim 11, further comprising at least one secondary ingredient selected from the group consisting of: oxa acid, oxa diacid, sunscreen, vitamin E, vitamin E acetate, vitamin C, vitamin C palmitate, kelp sea extract, cyclic AMP, ivy glycolic extract, willow bark extract, oat beta glucan, hops extract, fennel extract, milk extract, jojoba oil, ginseng extract, and any combinations thereof.

15. The composition of claim 14, wherein the sunscreen is selected from a group consisting of oxybenzone, sulsiobenzone, dioxybenzone, menthyl anthranilate, para aminobenzoic acid (PABA), DEA methoxycinnamate, octocrylene, octyl methoxycinnamate, octyl salicylate, homomenthyl salicylate, octyl dimethyl PABA, TEA salicylate, titanium dioxide, zinc oxide, butylmethoxy dibenzoylmethane, 4-methyl benzilidene camphor, octyl triazone, terephthalydidene dicamphor sulfonic acid and salts thereof, ethyl PABA, 2-(2′hydroxy-5′-methylphenyl) benzotriazole, methylene bis-benzotriazolyl tetramethylbutylphenol, phenylbenzimidazole sulfonic acid, bis-octoxyphenol methoxphenyl triazine, and any combinations thereof.

16. A method for providing an improvement in the aesthetic appearance of skin, hair and/or nails comprising applying thereto the composition of claim 11.

17. The method of claim 16, wherein the composition is applied to the skin and the improvement is selected from the group consisting of:
reducing dermatological aging;
decreasing skin fragility;
ameliorating and/or reversing loss of collagen and/or elastin;
ameliorating skin atrophy;
improving skin firmness/plumpness;
improving skin texture;
reducing the appearance/depth of lines and wrinkles;
improving skin tone/tautness;
decreasing pore size;
restoring skin luster/brightening;
minimizing signs of fatigue;
improving skin barrier function;
minimizing skin dryness;
reducing or ameliorating hyperpigmentation;
promoting/accelerating cell turnover;
enhancing skin thickness;
improving the appearance of skin contour;
increasing skin resiliency; and
minimizing skin discoloration.

18. The method of claim 17, wherein the improvement is selected from the group consisting of:
ameliorating and/or reversing loss of collagen and/or elastin;
ameliorating skin atrophy;
improving skin firmness/plumpness;
improving skin texture;
reducing the appearance/depth of lines and wrinkles;
improving skin tone/tautness;
enhancing skin thickness; and
increasing skin resiliency.

19. A composition comprising a cosmetically acceptable vehicle and, based upon the total weight of the composition,
about 0.001 wt % to about 1 wt % of a sunflower seed extract;
about 0.001 wt % to about 0.1 wt % of a pomegranate extract;
about 0.001 wt % to about 0.01 wt % of a soy bean extract;
about 0.001 wt % to about 0.1 wt % of a grape extract;
about 0.001 to about 0.1 wt % of panthenol;
about 0.001 wt % to about 0.01 wt % of hyaluronic acid;
about 0.001 to about 1 wt % phytol, isophytol or a combination thereof; and
about 0.01 wt % to about 0.1 wt % cucumber extract.

20. The composition of claim 19, further comprising at least one secondary ingredient selected from the group consisting of: oxa acid, oxa diacid, sunscreen, vitamin E, vitamin E acetate, vitamin C, vitamin C palmitate, kelp sea extract, cyclic AMP, ivy glycolic extract, willow bark extract, oat beta glucan, hops extract, fennel extract, milk extract, jojoba oil, ginseng extract, and any combinations thereof.

21. A method of providing an improvement in the aesthetic appearance of skin, hair and/or nails comprising applying thereto the composition of claim 19.

22. The method of claim 21, wherein the composition is applied to the skin and the improvement is selected from the group consisting of:
reducing dermatological aging;
decreasing skin fragility;
ameliorating and/or reversing loss of collagen and/or elastin;
ameliorating skin atrophy;
improving skin firmness/plumpness;
improving skin texture;
reducing the appearance/depth of lines and wrinkles;
improving skin tone/tautness;
decreasing pore size;
restoring skin luster/brightening;
minimizing signs of fatigue;
improving skin barrier function;
minimizing skin dryness;
reducing or ameliorating hyperpigmentation;
promoting/accelerating cell turnover;
enhancing skin thickness;
improving the appearance of skin contours;
increasing skin resiliency; and
minimizing skin discoloration.
